(19) United States
(12) Patent Application Publication
(10) Pub. No.: US 2022/0192987 A1
(43) Pub. Date: Jun. 23, 2022

(54) MICROCAPSULES CONTAINING CURCUMIN, AND METHODS FOR THE PRODUCTION THEREOF

(71) Applicant: NUABIOME LIMITED, Dublin (IE)

(72) Inventors: Sinead BLEIEL, Cork (IE); Robert KENT, Cork (IE); Olga MORENA MARRO, Cork (IE)

(73) Assignee: NUABIOME LIMITED, Dublin (IE)

(21) Appl. No.: 17/604,592

(22) PCT Filed: Mar. 20, 2020
(86) PCT No.: PCT/EP2020/057883

§ 371 Date: Oct. 18, 2021

(30) Foreign Application Priority Data
Mar. 20, 2019 (GB) 1903787.8

(51) Int. Cl. A61K 9/16

(52) U.S. Cl. CPC A61K 9/1652; A61K 9/1658

(57) ABSTRACT

“Microcapsules containing curcumin, and methods for the production thereof” A microcapsule suitable for delivery intact to the mammalian lower intestine via an oral route and having curcumin homogenously distributed in pockets within a crosslinked functional matrix, in which the microcapsule comprises at least 30% curcumin (w/w). Matrices formed of pea protein or Shellac have been proposed, using both spray-drying and cold gelation methods. Microcapsules of the invention are resistant to degradation in the stomach and release curcumin slowly in the ileum, and exhibit improved resistance of encapsulated curcumin to thermal and high/low pH environments.

### FIELD OF THE INVENTION

The present invention relates to curcumin containing microcapsules, compositions containing the microcapsules, and a method of making microcapsules containing curcumin.

### BACKGROUND TO THE INVENTION

Curcumin corresponds with the main active compound of Turmeric extract. Its health benefits (antioxidant, anti-inflammatory, anticarcinogenic, antibacterial, hypocholesterolaemia, etc.) are widely reported and scientifically proven. However, Curcumin has important technical drawbacks that impede its application as a nutraceutical and avoid mammals from eliciting its strong health benefits:

The incorporation of Curcumin in hydrophilic solutions is hindered by its very low water solubility. Even at low concentrations (0.04%), Turmeric extract (source of Curcumin) agglomerates preventing the formation of homogeneous solutions and causing precipitation and sedimentation when left to settle. This is a major drawback for its application as food ingredient or nutraceutical in the food industry (See FIGS. 10 and 11). The application of Curcumin in general food processes is also greatly hindered by its light sensitivity, chemical and thermal degradation. As a consequence of these poor properties, the suitability of Curcumin for long-term storage food products can be compromised.

Curcumin is sensitive to pH degradation. While it shows a very low solubility at low and neutral pH, it can dissolve at high pH. However, the increase of pH over neutrality also causes its degradation. This poor stability avoids the enhancement of its solubility (See FIG. 8) and again present a significant drawback for its application as a food ingredient or nutraceutical in the food industry.

The absorption of Curcumin under normal physiological conditions is severely limited due to its poor physico-chemical properties. While the low solubility will cause the presence of agglomerates, which cannot be absorbed, none of the physiological pH in the gastrointestinal tract (1.5 to 8) will help or assist Curcumin to become dispersed and absorbed. As a consequence, from the initial oral dose, a negligible amount of Curcumin will be available for absorption toward our bloodstream. This presents a huge issue for food formulators since Curcumin can't disperse easily in food products and pH causes various limitations leading to poor bioavailability and negligible benefits for a mammal.

Thermal treatment and oxidation of Curcumin causes its degradation by cleavage, leading to the generation of Ferulic acid and Vanillin. As a result, Curcumin shows a decrease in its main bioactive functions (See FIGS. 6 and 7). This degradation reduces its applicability in industry and domestic use, since a normal cooking and industrial sterilisation processes will induce the degradation of Curcumin.

WO2018/187849 and Birskey et al. (Birskey et al. (2018): Briskey, D., Sax, A., Mallard, A. R., & Rao, A. (2018); Increased bioavailability of curcumin using a novel dispersion technology system (LipiSperse®). European journal of nutrition, 1-11). describes a coating technology (HydroCurc) based in the utilization of surfactants, polar lipids and solvents which is claimed to increase the wettability and to prevent agglomeration of curcumin crystals. As a consequence, curcumin dispersibility is expected to increase, leading to an improved bioavailability of the active. While this technology provides for improved wettability, it does not protect the curcumin from aggressive environments involving elevated temperatures and excessive pH's. Moreover, the HydroCurc technology does not demonstrate gastric transit, gastric protection or intestinal bioavailability, and, in addition, employs synthetic ingredients (surfactants, polar lipids and solvents) that would limit product applications due to regulatory and labeling guidelines.

It is an object of the invention to overcome at least one of the above-referenced problems.

### SUMMARY OF THE INVENTION

The present invention addresses the need for a curcumin delivery system which protects curcumin during manufacture, storage, ingestion and gastro-intestinal delivery, and in particular protects curcumin against thermal destruction during food processing heat treatments (FIGS. 6 and 7), high pH environments (FIG. 8), and simulated digestion conditions (FIG. 9), and provides curcumin in a form that is stable and dispersible in an aqueous beverage (FIGS. 10 and 11). This is achieved by providing curcumin in a microencapsulated form by spray drying or cold gelation, in which the curcumin is contained and distributed in pockets throughout a crosslinked food grade matrix that is resistant to enzymatic erosion and physical break-up in the acidic condition of the stomach, and gets digested slowly in the ileum slowly releasing the curcumin with a steady and consistent kinetic-release profile. In addition, the Applicant has successfully provided microencapsulates that contain high amounts of curcumin (up to 65%). The Applicant has identified methods for making the microencapsulates of the invention, including a cold-gelation route in which the microencapsulates are produced by first extruding a mixture of the matrix material and curcumin through a nozzle to form microdroplets, and then gelating the microdroplets in a gelation bath, and a spray drying route. In addition, the Applicant has identified a number of different materials that can be used for the matrix, including food grade proteins and biopolymers optionally in combination with food grade gelling agents. The matrix of the present invention may be reinforced with the introduction of other active compounds, for example plant extracts (piperine, quercetin), oils, (glucose syrups, omega oils), vitamins (vitamin C, zinc, vitamin E, vitamin D, group B vitamins, etc.) and minerals (calcium, zinc, iron magnesium, etc.) may be incorporated into the matrix.

According to a first aspect of the present invention, there is provided a microcapsule suitable for delivery intact to the mammalian lower intestine via an oral route and having curcumin contained within a crosslinked matrix, in which the microcapsule generally comprises at least 30% curcumin (w/w).

In one embodiment, the matrix is thermally or chemically crosslinked. For example, when made by spray drying, the matrix is crosslinked by hardening in the hot air (thermal crosslinking), or when made by extrusion into a gelation bath, the pH of the bath and the presence of a hardening salt contribute to chemically crosslink the matrix.

In one embodiment, the microcapsule comprises at least 40%, 50%, or 60% or 65% curcumin (w/w) (i.e. intact curcumin).

In one embodiment, the crosslinked matrix is selected from a biopolymer, for example a food grade protein or resin, optionally combined with a food-grade gelling agent.

In one embodiment, the crosslinked matrix is formed from pea protein (or another food grade protein, such as a milk or plant protein) and/or a resin such as Shellac, typically in combination with a gelling agent optionally selected from alginate and pectin.

In one embodiment, the matrix material comprises denatured protein, shellac, or a combination of denatured protein and shellac.

In one embodiment, the microcapsule contains pockets of curcumin homogenously distributed throughout a continuous crosslinked matrix (i.e. multinuclear). Microcapsules of this form may be formed by spray drying or extrusion through a single nozzle into a gelation bath.

In another embodiment, the microcapsule has a core-shell morphology, and comprises a core of curcumin contained within a crosslinked matrix shell (i.e. mononuclear morphology). Microcapsules of this form may be formed by extrusion through a concentric double nozzle into a gelation both, in which the core is extruded through an inner nozzle, and the shell-forming matrix material is extruded through a concentric outer nozzle.

In one embodiment, the microcapsule has an average dimension of 20-150 μm using the method described below. At this size, the microcapsules have been found to be stable and dispersible in an aqueous solution at amounts of up to 0.5 g Curcumin per 100 ml. Microcapsules of this size range are generally produced by spray-drying.

In one embodiment, the microcapsule has a dimension of 150-300 μm using the method described below. Microcapsules of this size range are generally produced by extrusion and immersion into a hardening bath.

In one embodiment, the microcapsules are resistant to thermal degradation as determined by the thermal stability test below (Example 8).

In one embodiment, the microcapsules are resistant to pH degradation as determined by the pH stability test below (Example 9).

In one embodiment, the microcapsules are stable to settling in aqueous solution as determined by the settling stability test below (Example 11)

In one embodiment, the microcapsules are configured for slow release of the curcumin in the ileum.

In one embodiment, the microcapsules are formed by spray-drying or extrusion into a gelation bath. In one embodiment, extrusion may employ a single nozzle or a double nozzle.

In another aspect, the invention provides a composition comprising a multiplicity of microcapsules of the invention. The composition may be liquid, solid, semi-solid, an aerosol or a gel. The composition may be a food or beverage, or a nutritional supplement. In one embodiment, the composition is a powder.

In another aspect, the invention provides an aqueous composition comprising microcapsules of the invention stably dispersed within an aqueous vehicle. Typically, the microcapsules have an average dimension of 20-150 μm. Typically, the aqueous composition comprises about 0.1 to 1.0 g microcapsule per 100 mls aqueous composition. Stably dispersed means that not more than 20% of the dispersed microcapsules will settle out of the aqueous vehicle during a two-minute settling period following mixing for 5 minutes.

In another aspect, the invention provides a composition of the invention that has been heat-treated to 100° C. for 30 minutes in which at least 70% or 75% of the curcumin in the composition is intact following heat treatment. (i.e. not thermally degraded).

In another aspect, the invention provides a method of producing microcapsules having a curcumin contained within a crosslinked matrix, comprising the steps of:
mixing curcumin and a microcapsule matrix forming material selected from a food grade protein and/or a resin, optionally combined with a food-grade gelling agent; to form a dispersion
treating the dispersion to form microdroplets;
crosslinking the droplets to form microcapsules; and
drying the microcapsules.

In one embodiment, the method includes a step of homogenising the dispersion prior to the step of forming microdroplets.

In one embodiment, the method includes an initial step of forming a dispersion of matrix forming material, and then mixing the curcumin with the dispersion.

In one embodiment, the microdroplets are formed by extrusion and are crosslinked in a gelation bath comprising a hardening agent, for example a suitable salt. Examples include an ascorbate salt, or a calcium, magnesium, or zinc salt. Typically, the gelation bath is buffered to be acidic, for example a pH of 4.0 to 5.5. The gelation may comprise the hardening agent at 0.1 to 1.0 M, preferably about 0.5M. The gelation bath may comprise a surfactant, for example a TWEEN at 1.0 to 10%, typically about 5%. Extrusion may be through a single nozzle (mononuclear) or through a double nozzle (multinuclear).

In one embodiment, the microdroplets are formed and crosslinked in a spray dryer. In one embodiment, the inlet/outlet temperatures are about 150-170/60-90° C., preferably about 155-165/65-80° C., and ideally about 160/73° C.

In one embodiment, the dispersion comprises 5-15% protein and 0.5 to 5% gelling agent (w/v). In one embodiment, the dispersion comprises 3-7% curcumin (w/v). The protein may be pea protein. The gelling agents may be alginate or pectin.

In one embodiment, the dispersion comprises 15-35% resin and 0.5 to 5% gelling agent (w/v). In one embodiment, the dispersion comprises 5-8% curcumin (w/v). The resin may be shellac. The gelling agents may be alginate or pectin.

In one embodiment, the dispersion comprises a dispersion of protein, resin and curcumin. 4-6% protein, 4-6% resin (w/v). In one embodiment, the dispersion comprises 5-8% curcumin (w/v). The protein may be pea protein. The resin may be shellac. The gelling agents may be alginate or pectin.

In embodiments in which the matrix forming material, the method generally includes the steps of forming a protein dispersion (optionally in combination with a gelling agent), heating the dispersion sufficiently to denature the protein, adding curcumin, and then homogenising the dispersion. The protein is heated to provide at least 50% denaturation of the protein, i.e. at least 80% denaturation. A suitable heat treatment is 90° C. for 30 minutes.

In another aspect, the invention provides a method of forming a beverage comprising the steps of mixing 0.1 to 10%, 0.1 to 5.0%, 0.1 to 2.0% and preferably about 0.1 to 1.0% microcapsules (w/v) with an aqueous medium to form the beverage, wherein the beverage is stable to settling.

In one embodiment, the beverage is heat treated by pasteurisation or sterilisation, and in which at least 80%, 85% or 90% of the curcumin in the heat treated beverage remains active.

In another aspect, the invention provides a beverage formed according to the method of the invention.

In another aspect, the invention provides a method of forming a baked food product comprising the steps of mixing 0.1 to 10%, 0.1 to 5.0%, 0.1 to 2.0% and preferably about 0.1 to 1.0% microcapsules (w/v) with baked product ingredients (for example flour, sugar, butter etc) to form a mix, and baking the mix, in which at least 80%, 85% or 90% of the curcumin in the baked food product remains active.

In one embodiment, the mix is baked at a temperature of at least 100° C. for at least 20 or 30 minutes.

In another aspect, the invention provides a baked food product, optionally a cookie or muffin, formed according to the method of the invention.

In another aspect, the invention provides a composition of microcapsules formed according to the methods of the invention.

The technology of the present invention is based on the microencapsulation of curcumin extract and generation of microcapsules in which curcumin is entrapped in a functional matrix which provides important technological benefits:

i. Possibility of incorporation of synergetic ingredients, named plant extracts, antioxidant and anti-inflammatory compounds, vitamins, minerals, etc. Their incorporation in the matrix would allow to increase the nutritional value of curcumin microcapsules, enlarge the stability of the extract, enhance bioavailability, broaden the product applications in the food industry, etc.

ii. More robust protection against aggressive environment, such as thermal heating and pH degradation, due to the incorporation of curcumin into a protective shield provided by the matrix.

iii. Target delivery. The microencapsulation of curcumin according to the present invention provides resistance during the pass of curcumin throughout the gastrointestinal tract, to reach the intestinal villia properly dispersed for its absorption.

iv. Enhanced dispersibility proved by real quantification of stable microcapsules in aqueous medium and simulated intestinal fluid. Industry standards (WO 2018/187849 A1) offer enhanced dispersibility based in the reduction of the contact angle. While the contact angle provides an indirect or less exact measure of dispersibility, the present invention provides a real quantification of the curcumin in selected aqueous medium.

v. Enhanced bioavailability based in the improvement of dispersibility in simulated intestinal fluid and the incorporation to the formulation of technological coadjutants from natural origin to enhance the pharmacokinetic properties of curcumin. The present invention improves bioavailability providing a significantly higher dispersibility, enhanced absorption and metabolism. Industry standards (Birskey et al., 2018) base the enhanced bioavailability only in the dispersion improvement.

vi. The present invention is entitled to claim Clean label attribution. The ingredients incorporated into the matrix are known by consumers and recognised as wholesome. Industry standards (WO 2018/187849 A1) incorporate in their formulation surfactants, polar lipids and solvents that do not correspond with easy-to-recognise ingredients by consumers. This also represents a limitation in many areas as regulation, labelling, nutritional information and allergies.

Other aspects and preferred embodiments of the invention are defined and described in the other claims set out below.

### BRIEF DESCRIPTION OF THE FIGURES

FIG. 1. Light microscope images (50×) of curcumin extruded microcapsules obtained with sodium alginate and shellac.

FIG. 2. Light microscope image (50×) of curcumin spray dried microcapsules obtained with sodium alginate and shellac.

FIG. 3. Images of non-encapsulated curcumin (left) extract and curcumin microcapsules (right) obtained with pea protein and sodium alginate.

FIG. 4. Images of non-encapsulated curcumin extract (left) and curcumin microcapsules (right) obtained with sodium alginate and shellac.

FIG. 5. Images of non-encapsulated curcumin extract (left) and curcumin microcapsules (right) obtained with pea protein and shellac.

FIG. 6. Calibration curve of curcumin spray dried microcapsules obtained with sodium alginate and shellac.

FIG. 7. Effect of thermal treatment (100° C. during 30 min) on the load for non-encapsulated curcumin and curcumin spray dried microcapsules. Results expressed as percentage with respect to the initial curcumin load.

FIG. 8. Effect of thermal treatment (100° C. during 30 min) on the load for non-encapsulated curcumin and curcumin extruded microcapsules. Results expressed as percentage with respect to the initial curcumin load.

FIG. 9. Effect of high pH treatment (pH 9) under biological conditions (37° C. for 2 h) on the load for non-encapsulated curcumin and curcumin spray dried microcapsules. Results expressed as percentage with respect to the initial curcumin load.

FIG. 10. Concentration of curcumin with respect to the initial load after 2 h in simulated intestinal fluid. Results indicative of in vitro bioavailability. Non-encapsulated curcumin and and curcumin spray dried microcapsules.

FIG. 11. Image of aqueous beverage containing non-encapsulated curcumin (left) and curcumin spray dried microcapsules (right). 330 mL of beverage containing 500 mg of curcumin dose.

FIG. 12. Non-encapsulated curcumin and curcumin spray dried microcapsules stable in aqueous dispersion. Results expressed as percentage with respect to the initial curcumin load.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “microcapsule” or “microencapsulate”: means a polynuclear structure having an average dimension in the micron range, typically of 10-500 microns, 20-150 microns, or 150-300 microns, as determined using a method of laser diffractometery (Mastersizer 2000, Stable Micro Systems, Surrey, UK). This method determines the diameter, mean size distribution and D (v, 0.9) (size at which the cumulative volume reaches 90% of the total volume), of micro-encapsulates with diameters in the range of 0.2-2000 μm. For microencapsulate size analysis, micro-encapsulate batches were re-suspended in Milli-Q water and size distribution is calculated based on the light intensity distribution data of scattered light. Measurement of microencapsulate size is performed at 25° C. and six runs are performed for each replicate batch (Doherty et al., 20111) (Development and characterisation of whey protein micro-beads as potential matrices for probiotic protection, S. B. Doherty, V. L. Gee, R. P. Ross, C. Stanton, G. F. Fitzgerald, A. Brodkorb, Food Hydrocolloids Volume 25, Issue 6, August 2011, Pages 1604-1617). Preferably, the microencapsulate is substantially spherical. The microcapsules may be multinuclear (i.e. pockets of curcumin homogenously distributed throughout a continuous crosslinked matrix) or mononuclear (curcumin core contained within a crosslinked matrix shell).

As used herein, the term “Gastro-resistant”: means that the microencapsule can survive intact for at least 60 minutes in the simulated stomach digestion model described in Minekus et al., 1999 and 2014 (A computer-controlled system to simulate conditions of the large intestine with peristaltic mixing, water absorption and absorption of fermentation product, Minekus, M., Smeets-Peeters M, Bernalier A, Marol-Bonnin S, Havenaar R, Marteau P, Alric M, Fonty G, Huis in't Veld J H, Applied Microbiology Biotechnology. 1999 December; 53 (1):108-14) and (Minekus et al., 2014, A standardised static in vitro digestion method suitable for food—an international consensus, Minekus, A. et al., Food Function, 2014, 5, 1113).

As used herein, the term “Ileal-sensitive”: means that the microencapsulates are capable of releasing their contents in vivo in the ileum of a mammal.

As used herein, the term “protein” refers to any food grade protein, including plant derived protein (for example pea, soy and rice) or dairy protein (for example milk protein or fractions thereof including whey, casein and milk concentrates).

As used herein, the term “biopolymer” refers to any food grade biopolymer which is suitable for extrusion or spray drying and forming a crosslinked matrix. Examples include hydrocolloids solutions of different origins: protein such as milk or plant protein; polysaccharides such as alginate, pectin, carrageenan, modified cellulose, dextrines; biobased resins or shellac.

As used herein, the term “crosslinked” as applied to the matrix of the microencapsulates means that the biopolymer component of the matrix is crosslinked to harden the microencapsulate. Crosslinking may be chemical crosslinking, for example organic salt crosslinking (for example sodium ascorbate, sodium citrate or sodium acetate) optionally combined with a divalent inorganics salt crosslinking (for example calcium chloride or magnesium chloride, magnesium chloride or zinc chloride) that occurs in a gelation bath, or thermal crosslinking such as that which occurs due to heat in a spray dryer.

As used herein, the term “resistant to thermal degradation” as applied to a microcapsule of the invention should be understood to mean that at least 80%, and preferably at least 85% or 90%, (w/w) of the curcumin in an aqueous dispersion of microcapsules remains active after incubation at 100° C. for 30 minutes in the thermal stability test described below. The term “remains active” as applied to curcumin in the microcapsules means that the curcumin has not degraded to secondary compounds. The term “heat treated” or “thermal treatment” as applied to beverages containing the microcapsules of the invention generally means pasteurisation or an equivalent heat treatment (including HTST and LTST heat treatments) employed to destroy pathogens in food and extend shelf like.

As used herein, the term “resistant to pH degradation” as applied to a microcapsule of the invention should be understood to mean that at least 50%, and preferably at least 55%, (w/w) of the curcumin in an aqueous dispersion of microcapsules remains active after incubation in a 0.1 M sodium phosphate buffer with pH adjusted to 9 using 1M NaOH for two hours at 37 C in the pH stability test described below.

As used herein, the term “stable to settling in aqueous medium” as applied to the microcapsules of the invention means that not more than 20% of the dispersed microcapsules by weight will not settle out of the aqueous solution during a two-minute settling period in the settling stability test of Example 11. As used herein, the term “stable to settling” as applied to a beverage containing microcapsules (generally 0.1 to 1.0% w/v) of the invention means that not more than 20% of the dispersed microcapsules by weight will not settle out of the beverage during a two-minute settling period in the settling stability test of Example 11

As used herein, the term “spray drying” refers to methods of making microcapsules comprising preparing a spray-drying feedstock comprising an active agent (i.e. curcumin) and a matrix forming component, spraying (generally atomising) the feedstock into a drying chamber to produce microdroplets which are dried in the chamber to produce microcapsules, and collecting the microcapsules. In one embodiment, the inlet/outlet temperatures are about 130-150/60-90° C., preferably about 135-145/65-80° C., and ideally about 140/73° C.

As used herein, the term “cold gelation” refers to a process in which a mixture of active agent and matrix forming component is extruded through a nozzle to form microdroplets, which are collected in a gelation bath containing a hardening agent configured to crosslink the matrix forming material. Typically, the microbeads are cured in a gelation bath containing an acidic curing solution, suitably an acetate solution. Preferably, the parameters of the gelation bath are chosen to ensure instantaneous gelation of the microbeads. That is to say that the microcapsules gel (i.e. harden) immediately upon contact with the gelation bath. Generally, the parameters of the gelation that are varied are pH, acid concentration, and temperature. In the example provided below, in which a 9% pea protein or 25% shellac suspension is employed, and in which the pH-sensitive component is curcumin, the pH of the acidic solution is about 4.8, the sodium ascorbate concentration is about 0.4 to 0.6M, and the temperature is from 33° C. to 37° C. Typically, the gelation bath comprises a surfactant to prevent or inhibit agglomeration of the formed microbeads. Suitably, the surfactant is a Tween, ideally Tween 20. Suitably, the formed microbeads are subject to an extended curing period in the acidification bath, for a period of at least 15 minutes, and preferably for a period of at least 20 minutes. In a preferred embodiment of the invention, the formed microcapsules are cured for a period of time from 20 to 60 minutes in the bath.

A preferred method of producing the microdroplets is a vibrating nozzle technique, in which the suspension is sprayed through a nozzle and laminar break-up of the sprayed jet is induced by applying a sinusoidal frequency with a defined amplitude to the spray nozzle. Examples of vibrating nozzle machines are the ENCAPSULATOR (Innotech, Switzerland) and a machine produced by Nisco Engineering AG. Typically, the spray nozzle has an aperture of between 100 and 200 microns, preferably between and 140 and 160 microns, and ideally about 150 microns. Suitably, the frequency of operation of the vibrating nozzle is from 1150 to 1360 Hz. Generally, the electrostatic potential between nozzle and acidification bath is 0.9 to 1.25 V. Suitably, the amplitude is from 5.5 to 7. Typically, the falling distance (from the nozzle to the acidification bath) is less than 32 cm, preferably less than 40 cm, suitably between 20 and 40 cm, preferably between 25 and 35 cm, and ideally about 30 cm. The flow rate of suspension (passing through the nozzle) is typically from 2.0 to 3.5 ml/min. The nozzle may be a single nozzle for the production of multinuclear microcapsules, or a double concentric nozzle for the production of mononuclear microcapsules having a core-shell morphology, where the core is extruded through an inner nozzle and the shell matrix is extruded through an outer, concentric, nozzle.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

Microcapsules Made Using Cold Gelation Method with Protein Matrix

Pea protein isolate and sodium alginate are dispersed in RO water at 5-15% wt. and 0.5-5% wt., respectively. The dispersion is heat treated at 95° C. for 30 minutes to induce the denaturation of the protein.

Curcumin is incorporated to the dispersion in a final percentage of 3.5-6.5% wt. The dispersion is homogenised by using a Ultraturrax for 6 min (Ultra Turrax, IKA, Germany) to enhance the dispersibility of curcumin in the matrix.

Wet microcapsules are generated by extruding the dispersion containing curcumin throughout a vibrating nozzle (150-450 μm). The gelation is induced using an acidification bath comprised by the buffer of a sodium salt of an organic acid (0.1-1 M), and a divalent inorganic salt (0.1-1 M), adjusted to a final pH of 4.4-4.8.

Once generated, the wet microcapsules are collected from the gelation bath and then drying is conducted under vacuum by applying temperatures cycles from 60° C. to 40° C.

### Example 2

Microcapsules Made Using Cold Gelation Method with Shellac Matrix

Shellac solution (15-35% wt.) is incorporated with sodium alginate dispersed at 0.5-5% wt. The concentration of shellac and sodium alginate solids in the final dispersion is 3.5-4.5% wt and 1-2% wt., respectively.

Curcumin is incorporated to the dispersion in a final percentage of 5.5-7.5% wt. The dispersion is homogenised by using a Ultraturrax for 6 min (Ultra Turrax, IKA, Germany), to enhance the dispersibility of curcumin in the matrix.

Wet microcapsules are generated by extruding the dispersion containing curcumin throughout a vibrating nozzle (150-450 μm). The gelation is induced using an acidification bath comprised by the buffer of a sodium salt of an organic acid (0.1-1 M), and a divalent inorganic salt (0.1-1 M), adjusted to a final pH of 4.4-4.8.

Once generated, the wet microcapsules are collected from the gelation bath and then drying is conducted under vacuum by applying temperatures cycles from 60° C. to 40° C.

### Example 3

Microcapsules Made Using Cold Gelation Method and Protein-Shellac Matrix

Pea protein isolate is dispersed in RO water at a final concentration of 6-8% wt. The protein is then partially denatured at 55-75° C. to enhance the opening of the biopolymer chain. Pea protein dispersion is combined with a Shellac solution (15-35% wt.) to a final concentration of 4-6% wt for pea protein and 4-6% wt. for shellac.

Curcumin is incorporated to the dispersion in a final percentage of 5.5-7.5% wt. The dispersion is homogenised by using a Ultraturrax for 6 min (Ultra Turrax, IKA, Germany), to enhance the dispersibility of curcumin in the matrix.

Wet microcapsules are generated by extruding the dispersion containing curcumin throughout a vibrating nozzle (150-450 μm). The gelation is induced using an acidification bath comprised by the buffer of a sodium salt of an organic acid (0.1-1 M), and a divalent inorganic salt (0.1-1 M), adjusted to a final pH of 4.4-4.8.

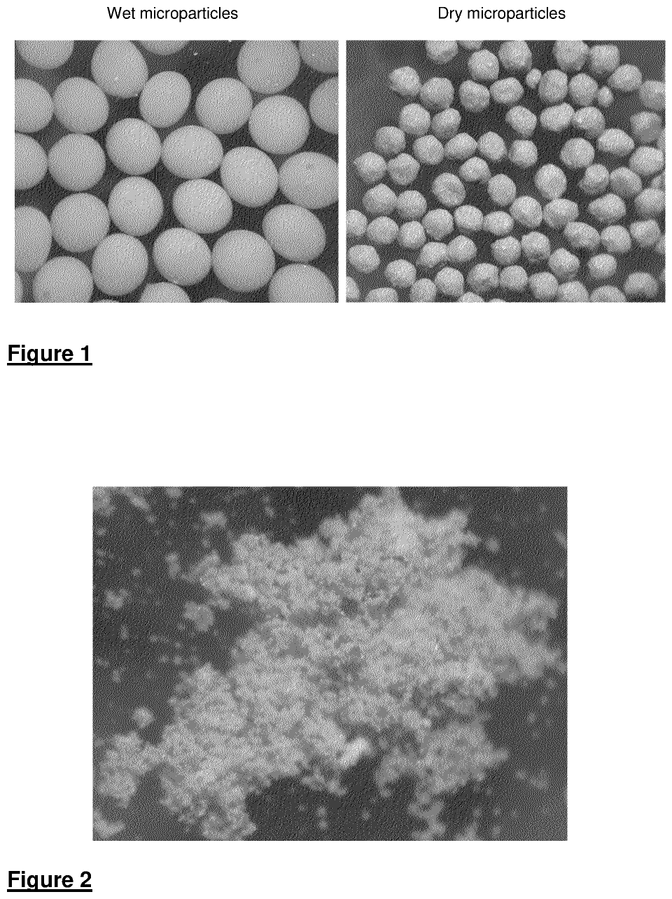

Once generated, the wet microcapsules are collected from the gelation bath and then drying is conducted under vacuum by applying temperatures cycles from 60° C. to 40° C. Results are shown in FIG. 1.

### Example 4

Microcapsules Using Spray Drying Method and Protein Matrix

Pea protein isolate and sodium alginate are dispersed in RO water at 5-15% wt. and 0.5-5% wt., respectively. The dispersion is heat treated at 95° C. for 30 minutes to induce the denaturation of the protein.

Curcumin is incorporated to the dispersion in a final percentage of 5.5-7.5% wt. The dispersion is homogenised by using a Ultraturrax for 6 min (Ultra Turrax, IKA, Germany), to enhance the dispersibility of curcumin in the matrix.

Dry microcapsules containing 30-40% wt. of curcumin are obtained by the generation of microdroplets by the atomization of the dispersion containing curcumin throughout a spray nozzle of about 1.7 mm of diameter. The flow rate for the dispersion is 8-12 mL/min. These generated microdroplets are died in contact with a hot air stream, adjusted to inlet/outlet temperatures of 130-150/65-85° C. Results are shown in FIG. 3.

### Example 5

Microcapsules Using Spray Drying Method and Shellac Matrix

Shellac solution (15-35% wt.) is incorporated with sodium alginate dispersed at 0.5-5% wt. The concentration of shellac and sodium alginate solids in the final dispersion is 3.5-4.5% wt and 1-2% wt., respectively.

Curcumin is incorporated to the dispersion in a final percentage of 5.5-7.5% wt. The dispersion is homogenised by using a Ultraturrax for 6 min (Ultra Turrax, IKA, Germany), to enhance the dispersibility of curcumin in the matrix.

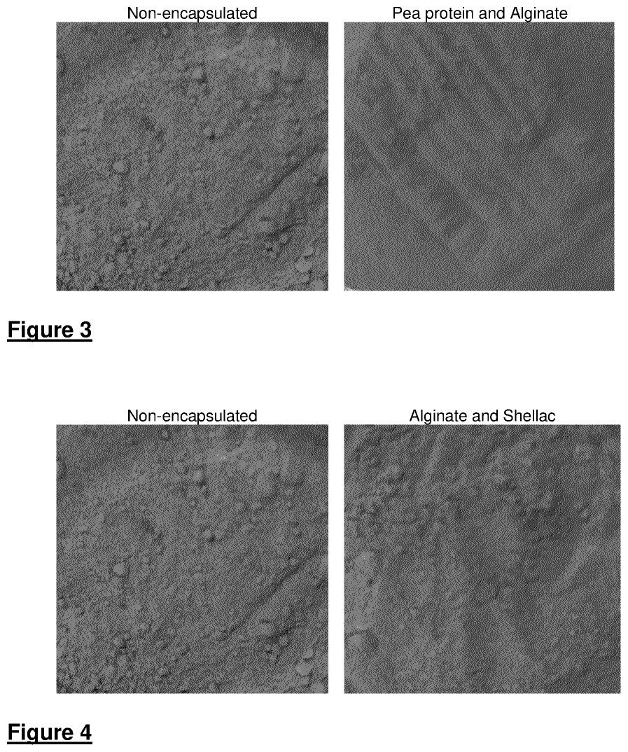

Dry microcapsules containing 45-55% wt. of curcumin are obtained by the generation of microdroplets by the atomization of the dispersion containing curcumin throughout a spray nozzle of about 1.7 mm of diameter. The flow rate for the dispersion is 8-12 mL/min. These generated microdroplets are died in contact with a hot air stream, adjusted to inlet/outlet temperatures of 130-150/65-85° C. Results are shown in FIGS. 2 and 4.

### Example 6

Microcapsules Using Spray Drying Method and Pea Protein-Shellac Matrix

Pea protein isolate is dispersed in RO water at a final concentration of 6-8% wt. The protein is then partially denatured at 55-75° C. to enhance the opening of the biopolymer chain. Pea protein dispersion is combined with a Shellac solution (15-35% wt.) to a final concentration of 4-6% wt for pea protein and 4-6% wt. for shellac.

Curcumin is incorporated to the dispersion in a final percentage of 5.5-7.5% wt. The dispersion is homogenised by using a Ultraturrax for 6 min (Ultra Turrax, IKA, Germany), to enhance the dispersibility of curcumin in the matrix.

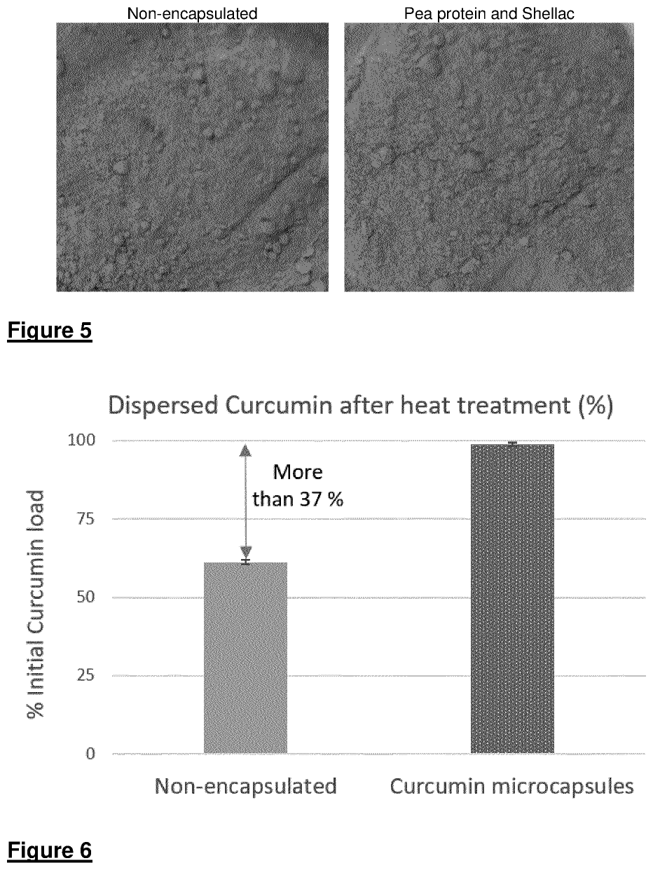

Dry microcapsules containing 45-55% wt. of curcumin are obtained by the generation of microdroplets by the atomization of the dispersion containing curcumin throughout a spray nozzle of about 1.7 mm of diameter. The flow rate for the dispersion is 8-12 mL/min. These generated microdroplets are died in contact with a hot air stream, adjusted to inlet/outlet temperatures of 130-150/65-85° C. Results are shown in FIG. 5.

### Example 7

Curcumin Quantification

The methodology applied for the quantification of curcumin is based in the methods described by Hazra et al. (Int. J. Pharmacogn, 2, 127-130. 2015), Pawar et al. (Nat Prod Chem Res, 6(1). 2018) and Sing and Avupati (Journal of Young Pharmacists, 9(4), 491. 2017). When being solubilized in an alcohol solvent, such as methanol or ethanol, curcumin is able to absorb light at 425 nm. A calibration curve of curcumin extract is prepared in a concentration of 100 mg/L, using methanol HPLC grade as solvent. Stock I is used to obtain Stock II, working solution, at a concentration of 10 mg/L. The calibration curve is obtained in the range of 1 mg/L to 5 mg/L, obtaining the different solutions from the Stock II. Measurements are carried out in a UV-Visible single beam spectrophotometer HALO XB-10 (Dynamica Scientific Ltd., Kirkton Campus, Livingston, United Kingdom), using 10 mm matched quartz cuvettes.

Different calibration curves are obtained for each of the different matrices produced. Non-encapsulated extract is used as control, with its corresponding calibration curve. Results are shown in FIG. 6.

### Example 8

Bioactive Characterization of Curcumin

Curcumin is characterised in its antioxidant activity by applying the ABTS (2,20-azino-bis[3-ethylbenzothiazoline-6-sulphonic acid]) assay using Trolox (6-hydroxy-2,5,7,8-tetramethylchroman-2-carboxylic acid) as a standard, a vitamin E analog. This assay is a modification of the method described by Re et al. (1999). The reaction is based in the reduction of the previously oxidized ABTS•+ radical cation, a blue/green chromophore. For this purpose, a 7 mM solution of ABTS is prepared in miliQ water and allowed to react with 2.45 mM potassium persulfate solution for 16 h in the dark, to generate the blue chromophore ABTS•+. This solution is then diluted using methanol HPLC grade to obtain an initial absorbance of 0.70 (±0.02) at 734 nm (A₀).

Curcumin extract solutions are obtained from non-encapsulated curcumin and curcumin microcapsules, at a concentration of 3 and 4 mg/L, using methanol HPLC grade as solvent. 80 μL of the test solutions are added to 920 μL of ABTS•+, causing a drop in absorbance due to the reduction of the ABTS•+ radical mediated by curcumin, after 6 minutes of reaction in the dark.

A calibration curve for the standard Trolox is obtained to relate the percentage of absorbance reduction after 6 minutes of reaction with the concentration of the standard (0 mg/L to 50 mg/L). Final results are expressed as TEAC units (Trolox equivalents antioxidant activity), corresponding with the concentration of 80 μL of curcumin solution that generate the same antioxidant activity than 80 μL of Trolox at 0.1 mM.

Alternative methods can be used to characterize the bioactivity of curcumin microcapsules, DPPH• test based in the hydrogen-donating ability of the stable free DPPH• (Martins et al., 2013), ferric-reducing power and ferrous ion-chelating ability, as described by Chan et al. (2009).

### Example 8

Thermal Stability of Curcumin in Microcapsules

The methodology for testing the thermal stability of microcapsules containing curcumin is a modification of the methods described by Aniesrani et al. (2014) and Wang et al. (2009). Dry microcapsules are dispersed in RO water in a final concentration of 0.15% wt. of Curcumin. All samples are stirred at 250 rpm during 5 minutes to assure the achievement of the highest solubility. In order to induce the thermal degradation of curcumin, the temperature of the solution is increased up to 100° C. and maintained for 30 min. The heat treatment is stopped by introducing the solution on ice, then the solution is slightly stirred, and a sample is taken to quantify the curcumin degradation. The parameters temperature and time are adjusted with the aim of simulating an ordinary domestic or industrial cooking process. As a consequence of the thermal treatment, curcumin is degraded to secondary compounds as vanillin, ferulic acid, vanillic acid, etc.

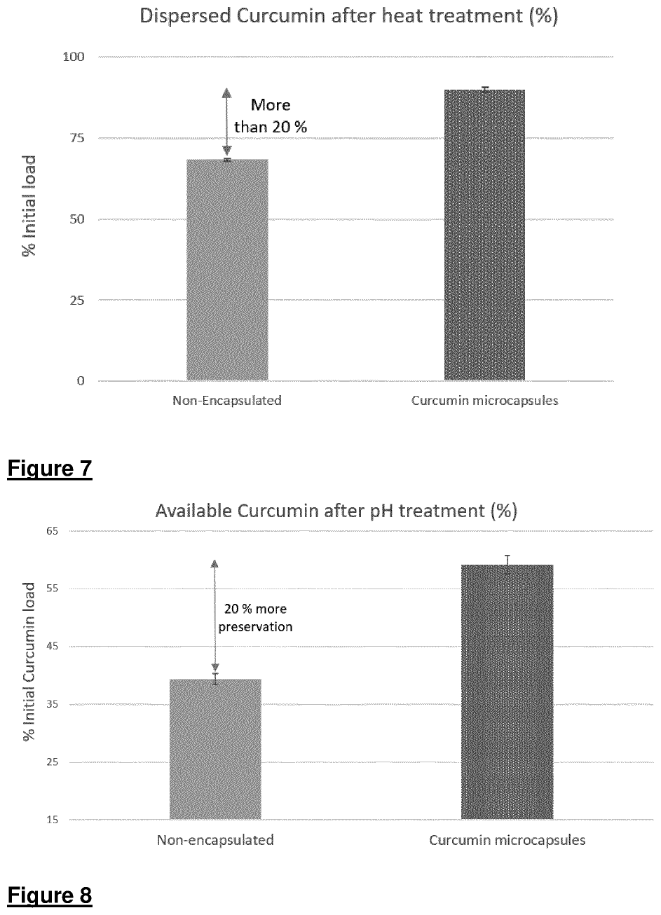

The quantification of curcumin is based in its light absorption ability at 425 nm, dissolved in an alcohol solvent as described in Example 7. Samples are diluted in order to measure in triplicate the absorbance of the curcumin concentrations 3 and 4 mg/L. The corresponding calibration curves to each formulations are used to calculate the real concentration of the sample. All tests are run in duplicate and non-encapsulated curcumin extract is used as control. Results are shown in FIGS. 7 and 8.

### Example 9

pH Stability of Curcumin in Microcapsules

The methodology for testing pH stability is a modification of the method described by Wang et al. (2009). Dry microcapsules are dispersed in 0.1 M sodium phosphate buffer with pH adjusted to 9 using 1M NaOH. The final concentration of Curcumin in solution is 0.15% wt. Samples are maintained under slight stirring (250 rpm) for two hours at 37° C., to simulate the physiological conditions, maintaining the pH stable at 9 during the assay. While curcumin solubility increases above neutrality, it also gets more sensitive to chemical degradation.

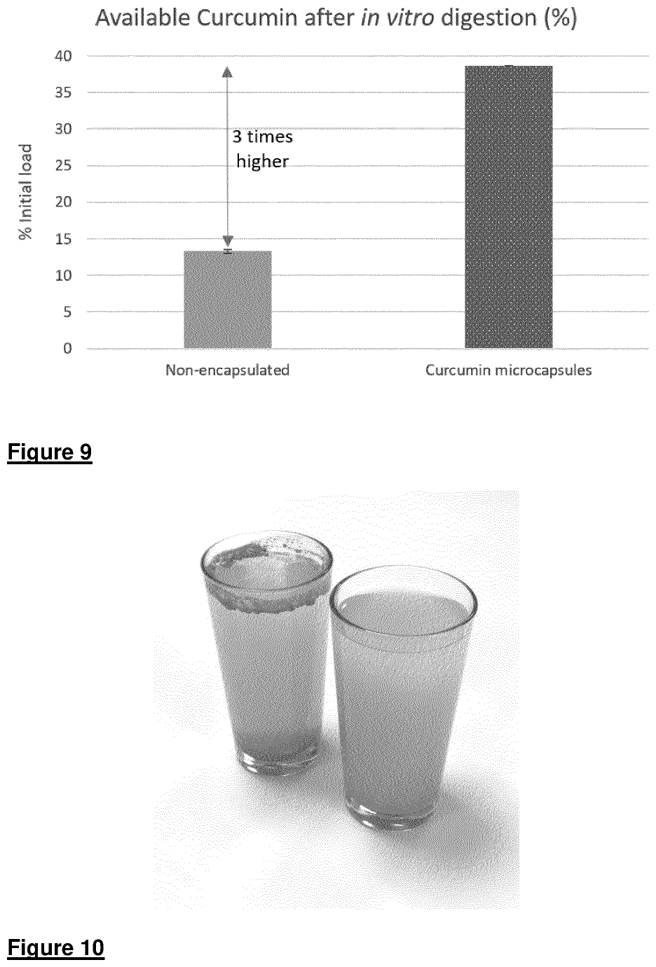

After 2 h exposed to a high pH, curcumin concentration is calculated based in its light absorption ability at 425 nm, dissolved in an alcohol solvent. Samples are diluted in order to measure in triplicate the absorbance of the curcumin concentrations 3 and 4 mg/L. The corresponding calibration curves to each formulations are used to calculate the real concentration of the sample. All tests are run in duplicate and non-encapsulated curcumin extract is used as control. Results are shown in FIG. 9.

### Example 10

In-Vitro Digestion Test

Dry microcapsules of curcumin are subjected to an in vitro digestion process to study the slow release of the polyphenol to physiological fluids. The methodology applied is based in the methods described by Al-Gousous and Langguth (Dissolution Technologies, 22(3), 6-9. 2015) and Minekus et al. (2014). With this aim, simulated saliva fluid (as per Minekus et al. 2014), simulated gastric fluid (as per Al-Gousous and Langguth, 2015) and simulated intestinal fluid (as per Al-Gousous and Langguth, 2015) are used. Oral phase is carried out for 2 minutes in the presence of salivary amylase; the gastric phase is prolonged for two hours, with the addition of pepsin; the digestion is finished with 2 hours of intestinal phase, in the presence of amylase, trypsin, chymotrypsin, pancreatin and bile.

The concentration of curcumin at different stages of the digestion process is calculated based in its light absorption ability at 425 nm, dissolved in an alcohol solvent. All tests are run in duplicate and non -encapsulated curcumin extract is used as control. Results are shown in FIG. 10.

### Example 11

Stability of Dispersed Microcapsules in Aqueous Medium (i.e. Stable from Settling)

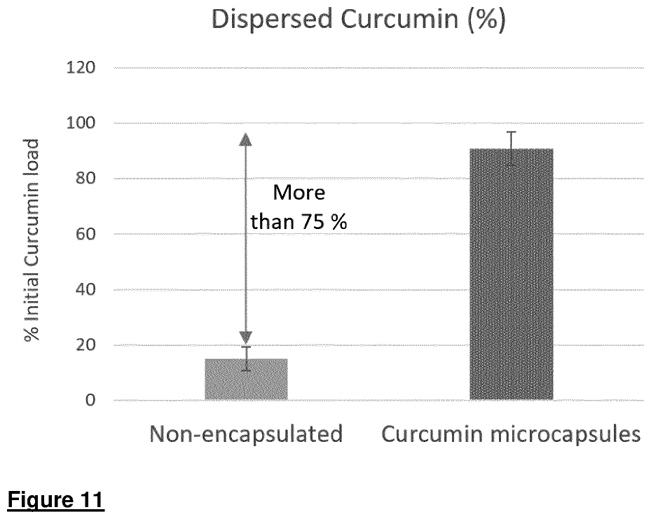

The methodology for demonstrating the stability of dispersed microcapsules in aqueous medium (i.e. stable from settling) is a modification of the method described by Wang et al., 2009. Dry microcapsules are dispersed in RO water in a final concentration of 0.15% wt. of Curcumin. Samples are stirred at 250 rpm for 5 min and then left to settle for 2 min. The concentration of curcumin in solution which does not generate precipitate, is calculated based in its light absorption ability at 425 nm, dissolved in an alcohol solvent, methanol HPLC grade. With this aim, samples are diluted in order to measure in triplicate the absorbance of the curcumin concentrations 3 and 4 mg/L. The corresponding calibration curves to each formulations are used to calculate the real concentration of the sample. All tests are run in duplicate and non-encapsulated curcumin extract is used as control. Results are shown in FIGS. 11 and 12.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A microcapsule suitable for delivery intact to the mammalian lower intestine via an oral route and having curcumin protected and stabilised in pockets within a functional gastric-resistant and ileal sensitive continuous crosslinked matrix, in which the microcapsule is resistant to thermal and pH degradation, stable to settling in aqueous medium, and comprises at least 30% curcumin (w/w).

2. A microcapsule according to any preceding claim in which the pockets of curcumin are homogenously distributed throughout the continuous crosslinked matrix.

3. A microcapsule according to claim 2, which is a spray dried microcapsule in which the matrix is thermally crosslinked.

4. A microcapsule according to claim 2, which is a cold gelated microcapsule in which the matrix is thermally crosslinked.

5. A microcapsule according to any preceding claim in which the matrix comprises:
denatured food grade protein and food grade gelling agent;
shellac and food grade gelling agent; or
denatured protein and shellac.

6. A microcapsule according to any preceding claim in which the matrix comprises denatured food grade protein and food grade gelling agent.

7. A microcapsule according to any preceding claim which is a spray dried microcapsule in which the matrix is thermally crosslinked and in which the matrix comprises denatured food grade protein and food grade gelling agent.

8. A microcapsule according to any preceding claim comprising at least 50% intact curcumin (w/w).

9. A microcapsule according to any preceding claim, in which the microcapsule has an average dimension of 20-150 μm.

10. A powder consisting of microcapsules according to any of claims 1 to 9, in which the powder is stable to settling in aqueous medium and in which at least 80% or 90% of the curcumin in the powder (w/w) remains active.

11. A beverage containing 0.1 to 1.0% microcapsules (w/v) according to any of claims 1 to 9.

12. A beverage according to claim 11 which is pasteurised or sterilised and in which at least 80% (w/w) of the curcumin in the microcapsules remains active.

13. A beverage according to claim 11 which is pasteurised or sterilised and in which at least 90% (w/w) of the curcumin in the microcapsules remains active.

14. A baked food product containing 0.1 to 1.0% (w/w) microcapsules according to any of claims 1 to 9 in which at least 80% (w/w) of the curcumin in the microcapsules remains active.

15. A method of producing microcapsules having curcumin contained within a crosslinked matrix, comprising the steps of:
mixing curcumin and a microcapsule matrix forming material selected from a food grade protein and/or a resin, optionally combined with a food-grade gelling agent; to form a dispersion
treating the dispersion to form microdroplets;
crosslinking the droplets to form microcapsules; and
drying the microcapsules.

16. A method according to claim 15, in which the microdroplets are formed and thermally crosslinked in a spray dryer.

17. A method according to claim 15, in which the microdroplets are formed by extrusion and chemically crosslinked in a gelation bath comprising a hardening agent.

18. A method according to any of claims 15 to 17, including a step of homogen ng the dispersion prior to the step of forming microdroplets.

19. A method according to any of claims 15 to 18, including an initial step of forming a pre-dispersion of matrix forming material, and then mixing the curcumin with the pre-dispersion to form the dispersion.

20. A method according to any of claims 15 to 19, in which the dispersion comprises 5-15% denatured food grade protein, 0.5 to 5% food grade gelling agent, and 3-7% curcumin (w/v).

21. A method according to any of claims 15 to 19, in which the dispersion comprises 15-35% shellac, 0.5 to 5% gelling agent and 5-8% curcumin (w/v).

22. A method according to any of claims 15 to 19, in which the dispersion comprises 4-6% denatured food grade protein, 4-6% shellac, and 5-18% curcumin (w/v).

23. A microcapsule formed according to the method of any of claims 15 to 22.

24. A method of forming a beverage comprising the steps of mixing 0.1 to 1.0% microcapsules (w/v) with an aqueous medium to form the beverage, wherein the beverage is stable to settling.

25. A method according to claim 24, in which the beverage is heat treated by pasteurisation or sterilisation, and in which at least 80% of the curcumin in the heat treated beverage remains active.

26. A beverage formed according to the method of any of claims 24 to 25.

27. A method of forming a baked food product comprising the steps of mixing 0.1 to 1.0% microcapsules (w/v) with baked product ingredients to form a mix, and baking the mix, in which at least 80% of the curcumin in the baked food product remains active.

28. A method according to 27, in which the mix is baked at a temperature of at least 100° C. for at least 20 minutes.

29. A baked food product, optionally a cookie or muffin, formed according to the method of any of claims 24 to 25.

30. A spray dried microcapsule suitable for delivery intact to the mammalian lower intestine via an oral route and having curcumin protected and stabilised in pockets within a functional gastric-resistant and ileal sensitive continuous thermally crosslinked matrix, in which the microcapsule has a dimension of 20-150 μm, is resistant to thermal and pH degradation, stable to settling in aqueous medium, and comprises at least 30% curcumin (w/w), in which the matrix comprises:
denatured food grade protein and food grade gelling agent; or
shellac and food grade gelling agent or denatured food grade protein.

31. A spray dried microcapsule according to claim 30 in which the protein is pea or milk protein and the gelling agent is alginate or pectin.

32. A heat treated beverage comprising 0.1 to 1.0% (w/v) of spray dried microcapsules of claim 30 or 31, wherein the beverage is stable to settling, and in which at least 80% of the curcumin in the heat treated beverage remains active.

33. A thermally treated beverage according to claim 32 in which the heat treatment is selected from pasteurisation and sterilisation.

34. A baked food product comprising 0.1 to 1.0% (w/v) of spray dried microcapsules of claim 30 or 31, wherein at least 80% of the curcumin in the spray dried microcapsules remains active.
